(19) United States
(12) Patent Application Publication
(10) Pub. No.: US 2004/0192976 A1
(43) Pub. Date: Sep. 30, 2004

(54) Process for hydrolyzing water-insoluble epoxides

(76) Inventors: Shaorong Chen, Wilmington, DE (US); Yanhui Sun, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/396,084

(22) Filed: Mar. 24, 2003

(51) Int. Cl. C07C 33/28

(52) U.S. Cl. 568/811; 568/867

(57) ABSTRACT

The invention relates to a process for hydrolyzing water-insoluble epoxides, and more particularly to a process for preparing diols. Specifically disclosed is a method for preparing 4-vinylcyclohexane-1,2-diol, 1,2-cyclohexane diol and 1,2-cyclododecane diol.

### FIELD OF THE INVENTION

Disclosed herein is a process for hydrolyzing water-insoluble epoxides at ambient conditions to produce the corresponding diols.

### BACKGROUND OF THE INVENTION

Polyesters and polyamides have excellent fiber properties, however, polyesters, especially polyester fibers are difficult to dye. Polyamides are not as difficult to dye as polyesters, but one or more dyeability additives are commonly incorporated into polyamides in order to selectively increase the affinity of the polyamide fibers for certain type of dyes or the resistance of the fibers to staining with certain type of staining agents. Compared to conventional cationic dyeability modifiers, 3-(2-sulfoethyl) hexanedioic acid, sodium salt (SEHA) confers improved basic dyeability to both polyesters and polyamides. A precursor to SEHA is 4-vinylcyclohexane-1,2-diol (VCH diol).

U.S. Pat. No. 6,312,805 B1describes a method to produce VCH diol by treating 4-vinylcyclohexene with formic acid and hydrogen peroxide and hydrolyzing the subsequent compounds with sodium hydroxide solution. It would be desirable to have a simpler and more economic process to produce VCH diol.

There have been many disclosures related to the hydration of water-soluble epoxides to prepare the corresponding diols. U.S. Pat. No. 4,165,440 discloses the hydrolysis of ethylene oxide to prepare ethylene glycol using acid ion exchange resin containing fluorinated alkyl sulfonic acid groups, at a temperature range of about 20° C. to about 115° C. JP04046133 describes the preparation of cyclododecane diol using cyclohexene oxide with water in the presence of inorganic solid acid catalysts at about 70° C.

There have also been disclosures of hydroylsis of water-insoluble epoxides using exchange resin materials. For example, U.S. Pat. No. 5,488,184 and U.S. Pat. No. 5,874,653 disclose propylene oxide in water using anionic exchange resin. The use of cation exchange resin (KU-23) to hydrolyze 2-butene oxide to 2,3-butanediol was published in Zhurnal Prikladnoi Khimii, Vol. 57, pp 2581-2584, 1984. The reaction was carried out at 50° C. with 10 mass % of KU-23, and 1:2 molar ratio of 2-butene oxide to water.

U.S. Pat. No. 3,062,889 describes the process for purifying acetaldehyde containing olefin oxides using a cation exchange resin (Amberlite IR-120) at a temperatures of 150° F. to 230° F. in which the olefin oxides was converted to glycols. U.S. Pat. No. 4,107,221 describes the chlorinated solvent purification process of using a strong acid cation exchange resin (Dowex, H⁺ form) in the presence of water to remove olefin oxides including propylene oxide, epichlorohydrin, glycidol, butylene oxide, cyclohexene oxide, and styrene oxide at temperatures from ambient to 165° C.

Several published reports mention that the cation exchange resins are used to convert epoxides to glycols with or without solvents (Monatsh Chem. page 745, 123, 1992; Monatsh Chem. Page 152, 122, 191; J. Med. Chem. Page 1334, 30, 8, 1987; Synthesis page 902,1996; J. Chem. Soc. Perkin Trans.1, page 742, 7, 1993; J. Chem. Soc. Perkin Trans.1 pages 1560 and 1562, 12, 1994). However, there is no prior art that discloses the hydrolysis of water-insoluble epoxides to prepare the corresponding diol, in the absence of a solvent, under ambient conditions, using a solid acid catalyst or ion exchange resin.

It would be desirable to have a process for the hydration of water-insoluble epoxides that does not require solvent, or the cost of energy to provide heat. The present invention presents such a process.

### SUMMARY OF THE INVENTION

This invention describes a process for hydrolyzing a water-insoluble epoxide of the general formula

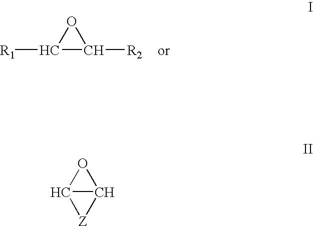

wherein, R₁ and R₂ independently are selected from the group consisting of C3 or higher saturated or unsaturated alkyl or aryl groups, which are substituted or unsubstituted, and wherein Z is C3 or higher saturated or unsaturated alkyl or aryl groups, which are substituted or unsubstituted;

said process comprising:

contacting the water-insoluble epoxide with water in the presence of a solid acidic catalyst or an ion exchange resin at ambient conditions for a time sufficient to produce the corresponding diol.

### DETAILED DESCRIPTION OF THE INVENTION

The present invention discloses a process for preparing diols from water-insoluble epoxides. 4-vinylcyclohexene-1,2-epoxide (represented by compound 1) was hydrolyzed to prepare 4-vinylcyclohexene-1,2-diol (represented by the compound 2) with cationic exchange resin or solid acidic catalyst in water at ambient conditions.

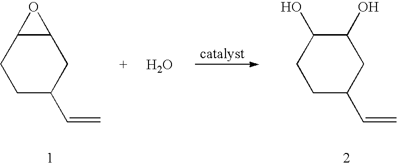

The epoxides that are useful in the present invention are those C3 to C12 substrates having an epoxide moiety. These include, but are not limited to of 4-vinylcyclohexene-1,2-epoxide, 1,2-cyclododecane epoxide and 1,2-cyclohexane epoxide. Although these epoxides are not soluble in water, the process of the present invention can be used without solvent and at ambient conditions.

One embodiment of the present invention is the hydrolysis of 4-vinylcyclohexene-1,2-epoxide to 4-vinylcyclohexene-1,2-diol using heterogeneous catalysts, in water, without solvent, and at ambient condition. The ability to carry out the process at ambient conditions reduces the production cost of diol dramatically, which in turn is used as a co-monomer for polyester and polyamide production.

Another embodiment of the present invention is the hydrolyses of 1,2-cyclododecane epoxide and 1,2-cyclohexane epoxide to their corresponding diols.

In the process of the present invention, a vessel is charged with a solid acid catalyst or ion exchange resin, water and a water insoluble epoxide. The mixture is agitated, preferable vigorously, at ambient conditions.

The solid acid catalysts that are suitable for the present invention are silica alumina and TiF₄/SiO₂. It has also been discovered that inexpensive ion exchange resins that are available for water treatment can be used to hydrolyze the epoxide with high conversion and yield.

The ion exchange resins that are suitable for the present invention are Amberlyst-15, Dowex-HGR-W2H, Dowex-M33, Dowex-MSC-1-H, Dowex M-31, and NAFION.

Generally, the quality of the water that can be used for the present process is not significant. Untreated water may be used. Deionized water is preferred to increase the catalyst activity. The molar ratio of water to epoxides is in the range of 8:1-20:1. The contact time is about 5 hours.

### EXAMPLES

Catalyst A: Duolite ES-26 (strong acid cation resin, sodium form) was treated with dilute sulfuric acid, followed by DI water to neutral.

Catalyst B: Amberlyst (strong acid cation resin) was washed with DI water to neutral.

Catalyst C: 20 g. Silica (380 m²/g) was dispersed in 250 ml DI water under stirring. 0.8 g. TiF₄ was added into the suspension. The pH of the suspension was adjusted by HCl solution to 8. The mixture was stirred at ambient condition. The gel was filtered and washed with DI water to neutral. The solid was dried in a 120° C. oven.

### Example 1

### Hydrolysis of 4-Vinylcyclohexene-1,2-epoxide

A 250 ml slurry reactor was loaded with the catalyst (25 g; 16 wt %), deionized water (100 g; 5.55 mol) and 4-vinylcyclohexene-1,2-epoxide (75 g; 0.6 mol). The mixture was vigorously stirred at ambient condition for 5 hours. An end of run sample was taken for analysis by NMR. The conversion of epoxide (compound 1) was >99%, and the yield of diol (compound 2) was >95%.

### Example 2

### Hydrolysis of 1,2-cyclohexne epoxide

1,2-cyclohexene epoxide (3.1 g, 0.027 mol), and deionized water (4 g, 0.2) and Amberlyst-15 (1 g, 12 wt %) were added into a 20 ml of glass vial with a magnetic stir. The mixture was stirred vigorously for 5 hours. The final product was trans-1,2-cyclohexanediol. The conversion of epoxide was >99%, and the yield of diol was >95%.

### Example 3

### Hydrolysis of 1,2-cyclododecane epoxide

1,2-cyclododecane epoxide (3.6 g, 0.02 mol), and deionized water (4 g, 0.2) and Nafion® SAC-13 (1 g, 12 wt %) were added into a 20 ml of glass vial with a magnetic stir. The mixture was stirred vigorously for 48 hours. The final product was trans-1,2-cyclododecane-1,2-diol. The conversion of epoxide was >50%, and the yield of diol was >95%.

What is claimed is:

1. A process for hydrolyzing a water insoluble epoxide of the general having the formula 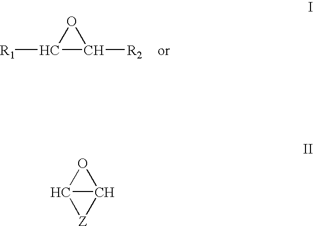
wherein, R₁ and R₂ independently are selected from the group consisting of C3 or higher saturated or unsaturated alkyl or aryl groups, which are substituted or unsubstituted, and wherein Z is C3 or higher saturated or unsaturated alkyl or aryl groups, substituted or unsubstituted;
said process comprising:
contacting the water insoluble epoxide with water in the presence of a solid acidic catalyst or an ion exchange resin at ambient conditions for a time sufficient to produce the corresponding diol.

2. The process of claim 1 wherein said water-insoluble epoxide is selected from the group consisting of 4-vinylcyclohexene 1,2-epoxide, 1,2-cyclohexane epoxide, and 1,2-cyclododecane epoxide.
